(19) United States
(12) Patent Application Publication
(10) Pub. No.: US 2024/0342265 A1
(43) Pub. Date: Oct. 17, 2024

(54) E PROTEIN-MUTATED WEST NILE VIRUS USED AS LIVEATTENUATED VACCINE AND ONCOLYTIC DRUG FOR CANCER THERAPY

(71) Applicant: SICHUAN ANCOCARE BIOPHARMACEUTICAL, Ltd., Chengdu (CN)

(72) Inventor: Li YU, Gaithersburg, MD (US)

(21) Appl. No.: 18/626,704

(22) Filed: Apr. 4, 2024

(30) Foreign Application Priority Data
Apr. 4, 2023 (CN) 202310349109.5

(51) Int. Cl. A61K 39/12; A61K 35/768; A61K 39/00; A61P 37/04; C07K 14/705; C12N 7/00

(52) U.S. Cl. CPC A61K 39/12; A61K 35/768; A61P 37/04; C07K 14/70532; C12N 7/00; A61K 2039/5254; A61K 2039/585; C12N 2770/24122; C12N 2770/24132; C12N 2770/24134; C12N 2770/24162; C12N 2770/24171

(57) ABSTRACT

The invention provides a recombinant West Nile virus, in which the amino acid sequence of envelope E protein is genetically modified to attenuation, and its RNA genome is inserted with a foreign gene fragment. The engineered E gene contains the mutation of five amino acids for reducing its neural virulence to the central nervous system; the integrated foreign gene between E and S1 gene makes a new chimerical virus. Thus, the present invention provides the application of this attenuated West Nile virus as a vaccine in preventive medicine and the application of the RNA-viral vector as a novel gene-drug in the pharmaceutical industry. The newly attenuated virus may fill the gap of no live-attenuated vaccine to the West Nile virus epidemic. The attenuated and recombinant virus can be used as an RNA oncolytic virus to target solid tumors, especially neural tumors, for cancer therapy with higher safety.

### REFERENCE TO AN ELECTRONIC SEQUENCE LISTING

The contents of the electronic XML sequence listing (File Name: 2950005sequencelisting.xml; Size: 4 kilobytes; and Date of Creation: Apr. 9, 2024) are herein incorporated by reference in entirety.

### CROSS-REFERENCE TO RELATED APPLICATION

This application claims the priority of Chinese Patent Application No. 202310349109.5, filed on Apr. 4, 2023, the content of which is incorporated herein by reference in its entirety.

### TECHNICAL FIELD

The present invention relates to the fields of preventive medicine and biopharmaceuticals. The genetically modified WNV can be applied to prevent WNV infectious diseases as a live-attenuated vaccine and treat cancers as a novel oncolytic immunotherapy drug. The genetically modified WNV has five amino acid mutations on the viral envelope € protein for attenuation and a foreign gene insertion in the viral genome to act as immunotherapy ingredients. The attenuated WNV lost its pathogenesis to CNS but kept its replication capability, and thus can be safely used for vaccination as a live vaccine and for anti-cancer drugs as an oncolytic virus.

### BACKGROUND

Within the Flaviviridae family, the Flavivirus genus comprises around 72 or more species of animal viruses. Of these, approximately 15 are pathogenic to humans, including the Yellow Fever Virus (YFV), Zika Virus (ZIKV), Dengue Virus (DENV), WNV, Japanese Encephalitis Virus (JEV), and Tick-borne Encephalitis Virus (TBEV), among others. Several common characteristics are shared among the Flaviviruses: most of these viruses are primarily transmitted through the bite of infected arthropods (mosquitoes or ticks), classifying them as arboviruses. Their genomes consist of positive-sense, single-stranded RNA; the viral particles are spherical with a diameter generally ranging from 40 to 70 nm, and they are enveloped with a lipid membrane surrounding their RNA. The viral particles contain three structural proteins (Capsid, M, and envelope). The envelope (E) protein plays a crucial role in infection and cell entry.

Within the Flavivirus genus, YFV-17D and JEV14-14-2 are approved attenuated-live vaccines that have been successfully used to prevent the outbreaks of Yellow Fever Virus (YFV) and Japanese Encephalitis Virus (JEV) diseases, respectively. Historically, outbreaks of West Nile Virus-induced WNV fever and encephalitis/meningitis diseases have occurred in various parts of the world. However, currently, no effective drugs or human vaccines are available against this virus infection. Developing an attenuated vaccine for WNV could play a positive role in preventing and halting the spread of this disease, contributing significantly to public health. A crucial aspect of vaccine safety is the absence of neurotoxicity in the viral vaccine. Our research indicates that the WNV pathogenicity and neurotoxicity are primarily associated with its surface envelope (E) protein. It is necessary to make the E protein mutation attenuation to develop WNV into a clinically applicable therapeutic for cancer or a preventive vaccine.

In the past decade, oncolytic virus drugs have emerged as a novel anti-tumor treatment, becoming an essential immunotherapeutic method. To date, only three oncolytic virus products have been approved for marketing worldwide, namely: 1. H101 (Oncorine), approved in China in 2003, is a recombinant human adenovirus type 5 with deletion of the E1B-55 kD and E3 gene fragments (78.3-85.8mu), used in the treatment of advanced nasopharyngeal carcinoma. 2. Imlygic (T-VEC/Talimogene), approved in the United States in 2015, a genetically modified herpes simplex virus type 1 (HSV-1) is used to treat melanoma. 3. Delytact (Teserpaturev, G47Δ), approved in Japan in 2021, a modified herpes simplex virus type 1 (HSV-1), is applied in treating glioma through a similar mechanism of oncolysis.

The development of the oncolytic virus indicates a growing recognition of the potential of oncolytic viruses as a therapeutic option for cancers, leveraging the ability of these viruses to selectively infect and kill tumor cells while stimulating an anti-tumor immune response.

The current market products and clinical research are mainly focused on DNA viruses, such as Herpes Simplex Virus (HSV), Vaccinia Virus, and Adenovirus (ADV), which are either commonly epidemic pathogens or extensively vaccinated. Studies have shown that over 70% of the global population is seropositive for HSV-1, and the overall proportion of the seropositive population for Ad5 (a type of adenovirus) is 85.2%. Thus, the pre-existing immunity within the body against these viruses can potentially hinder the effectiveness of viral therapies. When these oncolytic viruses are reintroduced into the body as therapeutic agents, the immune system's memory cells quickly recognize and mobilize related immune cells to eliminate or clear the virus, significantly reducing their therapeutic efficacy. Furthermore, even in patients without pre-existing immunity to the virus used in oncolytic therapy, the body will generate an immune response to the virus after the first administration. Antibodies produced in the short term can neutralize the virus, leading to a reduced duration of drug effectiveness and diminishing the effects of subsequent treatments. Therefore, overcoming the body's immune response to oncolytic viruses and finding ways to sustainably and effectively kill tumors is critical to the success of oncolytic virus therapy.

Another defect of oncolytic viruses as therapeutic agents is their inherent pathogenicity to humans. Early, non-genetically modified, and un-attenuated oncolytic viruses, such as Egypt 101 (a strain of West Nile virus), were tested in clinical trials for treating malignancies, including gastrointestinal, lymphosarcoma, bronchogenic carcinoma, ovarian, and breast cancers. Due to the pathogenic nature of the viruses, severe toxic side effects were observed in clinical treatments, which hindered further drug development using the oncolytic virus. Therefore, knocking out the viral genes responsible for pathogenicity to ensure the safety of oncolytic virus therapy is one of the essential conditions for the success of this treatment.

No reports of attenuated oncolytic WNV carrying exogenous active molecules used as clinical therapeutic drugs have been reported.

### SUMMARY

The objective of the present disclosure is to reduce the WNV's pathogenicity or toxicity to the CNS, making the attenuated WNV serve as a live vaccine capable of preventing WNV diseases. Meanwhile, the viral vector function is utilized by introducing exogenous genes into the viral genome to endow the virus with an immunotherapy function for clinical application. For example, using the attenuated virus for treatment as an oncolytic virus for cancer therapy can overcome the pathogenic side effects of oncolytic viruses, and it has high targeting efficiency and sound oncolytic effects.

The present invention adopts the following technical solutions to achieve the above object:

The invention provides an attenuated West Nile virus with envelope E protein mutation. It has at least 98% homology with the amino acid sequence shown in SEQ ID NO: 1. Moreover, compared with SEQ ID NO: 1, the amino acid sequence has amino acid substitutions at least at the 138th position, the 172nd position, the 173rd position, the 276th position, and the 312th position.

The present invention also provides a recombinant West Nile virus, wherein, compared with the sequence of the envelope E protein of the virus and SEQ ID NO: 1, the amino acid sequence of the envelope E protein has at least the following mutations at the same time:
(i) The 138th Glutamic acid E is replaced by Lysine K,
(ii) The 172nd Tyrosine Y is replaced by Valine V,
(iii) Threonine T at position 173rd is replaced with Alanine A,
(iv) The 276th Lysine K is replaced by Methionine M,
(v) Replacement of Alanine A at position 312th with Valine V,

Preferably, the sequence of the envelope E protein is the sequence shown in SEQ ID NO: 2.

Furthermore, it is emphasized that the sequence sites of the mutated five amino acids are consistent with SEQ ID NO: 1. Due to differences in the amino acid sequences of the E proteins of various WNV subtypes, there may be slight differences in the corresponding sequence numbers (sites) with other WNV subtypes.

Moreover, compared to the amino acid sequence and SEQ ID NO:1, it includes but is not limited to one or more amino acid substitutions at positions 138, 172, 173, 276, or 312. The amino acid sequence of the five sites on the E protein includes but is not limited to the following mutations or substitutions with similar structures and properties: achieving the genetic modification of the E protein, maintaining the infectivity of WNV but losing the ability to invade the CNS from the periphery.
(i) Glutamine (Glu/E) at position 138 is replaced with Lysine (Lys/K), arginine (Arg/R), or histidine (His/H) and other basic amino acids;
(ii) Tyrosine (Tyr/Y) at position 172 is replaced with Valine (Val/V), Methionine (Met/M), isoleucine (Ile/I), leucine (Leu/L), or Alanine (Ala/A);
(iii) Threonine (Thr/T) at position 173 is replaced with Alanine (Ala/A), Valine (Val/V), isoleucine (Ile/I), leucine (Leu/L), or Methionine (Met/M);
(iv) Lysine (Lys/K) at position 276 is replaced with Methionine (Met/M), Valine (Val/V), isoleucine (Ile/I), leucine (Leu/L), or Alanine (Ala/A);
(v) Alanine (Ala/A) at position 312 is replaced with Valine (Val/V), isoleucine (Ile/I), leucine (Leu/L), or Methionine (Met/M).

In addition, this invention applies the said attenuated WNV as a live attenuated vaccine to prevent infectious diseases caused by the virus.

This invention utilizes genetic engineering technology to alter the nucleotides of the WNV E protein sequences, resulting in 5 amino acid changes: Glutamine at position 138 to Lysine, Tyrosine at position 172 to Valine, Threonine at position 173 to Alanine, Lysine at position 276 to Methionine, and Alanine at position 312 to Valine. The attenuated WNV is thereby prevented from infecting the CNS through the blood-brain barrier. While it loses its ability to infect the CNS from the periphery, it can still replicate in neural tumor cell lines in humans and mice. Crucially, this attenuated virus retains the WNV antigenicity, effectively stimulating an immune response in the body, and is thus used for the preventive vaccination of WNV attenuated-live vaccines.

Currently, there are no approved WNV vaccines for human worldwide. The development of humans WNV attenuated-live vaccines focuses on mutations in the 3′-end non-coding region, including early nucleotide substitutions in the 3′-end region. An attenuated-live vaccine for West Nile virus was reported by replacement of the virulence gene sequence in the West Nile virus 3′ non-coding region (3′ UTR) with a polyadenylate poly (A) sequence.

There have been no reports of any attenuated-live vaccines by modifying the WNV E protein. Since the WNV E protein is the virus's surface protein, constituting the viral particles' physical structure, it is the major protein antigen of the virus. At the same time, the E protein carries multiple biological functions, including recognizing host cell receptors, envelope fusion, and entry into host cells. Therefore, amino acid mutations in the E protein might affect the packaging and release of the virus, thereby affecting the production of new viral particles and reducing infectivity, thus affecting the virus's invasiveness and pathogenicity to host cells. It can be modified into an attenuated-live vaccine strain by mutating the appropriate amino acids in the E protein to weaken the virus's pathogenicity (capability) while retaining its antigenicity and replication (infectivity). Our research has proven that excessive amino acid mutations in the E protein affect the virus's replication or survival, and amino acid mutations not related to neurotoxicity do not achieve neurotoxicity attenuation effects. The five amino acids of the E protein described in this invention have been experimentally proven to be related to the virus's neurotoxicity or critical amino acids for invading the CNS. Mutating these five amino acids as a cluster prevents WNV from infecting the CNS while retaining the virus's antigenicity. Therefore, it is an excellent attenuated-live vaccine for preventing infectious diseases caused by West Nile virus.

Our animal toxicology experiments also proved that WNV is very safe with mutations/substitutions of the five amino acids in the E protein. It did not cause infection and disease in sensitive animals.

Thirdly, this invention applies the attenuated West Nile virus as an RNA virus vector in the pharmaceutical field. Its E protein-attenuated virus vector can be embedded with exogenous (non-viral) genes and used as a gene therapy drug for clinical treatment.

Gene therapy as an application in clinical medicine has seen significant potential. Gene therapy must have a therapeutic target gene and a gene vector to introduce the therapeutic gene into cells. Therefore, gene vectors play a crucial role in gene therapy; they can accurately deliver therapeutic genes to target cells, thereby achieving disease treatment. Currently, gene vectors in gene therapy are mainly divided into two categories:

Viral vectors: These include lentivirus, adenovirus, retrovirus, adeno-associated virus, etc. Viral vectors have higher transduction efficiency, more robust engineering capabilities, and exceptionally high infection efficiency. Diseases are treated by directly delivering therapeutic genes into patients' bodies through the infectivity of viral vectors.

Additionally, when the virus serves as a vector, it is necessary to insert or integrate the target therapeutic gene into the viral gene vector while ensuring that the inserted exogenous gene can be transcribed and expressed without affecting the replication and biological activity of the viral vector itself. Due to this technical reason, currently, besides retroviruses, there have been no reports that members of the Flavivirus genus, including WNV, were used as gene therapy vectors.

At the same time, among the various factors that need to be considered in viral gene vectors in gene therapy, the virus's own pathogenicity and toxic effects also hinder the development of RNA virus vectors. Most RNA viruses are pathogenic and cannot be directly used for therapeutic purposes.

However, it has lost its pathogenicity since we have successfully attenuated WNV. Using this attenuated-live virus and unique genetic engineering technology, we have also successfully inserted exogenous genes into the WNV genome without affecting its replication and infectivity. Therefore, the WNV has been modified into an attenuated RNA virus vector to deliver therapeutic genes as therapeutic drugs.

In some specific embodiments of this invention, the insertion positions of the said exogenous non-viral genes include but are not limited to between the E gene and the non-structural NS1 gene.

In some specific embodiments of this invention, the said exogenous non-viral genes include but are not limited to T-cell co-stimulatory factors; preferably, the said T-cell co-stimulatory factors include but are not limited to CD86/CD80.

Fourthly, the attenuated WNV described in this invention is due to the loss of pathogenicity to the CNS. However, it retains its specific infectivity and ability to kill tumor cells in certain tissues. Therefore, it provides the attenuated WNV as an oncolytic virus drug for treating cancers.

Oncolytic viruses are viruses that can selectively infect and kill cancer cells. These viruses can directly destroy tumor cells and stimulate the host's anti-tumor immune system response. Currently, many different DNA and RNA oncolytic viruses are undergoing clinical trials. RNA oncolytic viruses include poliovirus, Coxsackievirus, togavirus, and VSV. However, no Flavivirus genus, including WNV oncolytic viruses, has been used in clinical cancer treatment or approved for marketing. Meanwhile, the attenuated WNV described in this invention can specifically target and kill neural tumors.

In some specific embodiments, the said attenuated WNV can infect and kill neural and other solid tumors. This invention uses attenuated WNV to design and develop RNA oncolytic viruses. This attenuated oncolytic virus can not only directly kill tumor cells but also insert exogenous gene fragments, such as 86CD/CD80, for tumor immunotherapy. Based on the immunological two-signal principle and the primary function of co-stimulatory molecules to induce T-cell immune activity, a synthetic RNA oncolytic virus drug (recombinant WNV-CD86, also known as DS1-H2-1) was constructed, by which the attenuated WNV was integrated with the human CD86 gene to act as an oncolytic-immunotherapy drug. This recombinant virus can infect tumor tissues and reproduce in cells to kill tumors (oncolysis) while ectopically expressing the CD86 protein molecule. The CD86 protein is a necessary signal molecule for activating immune T cells and is the “master switch” for immune B cells to regulate T cell activation and cellular immune response. Therefore, with the help of CD86 molecule expression in tumors through the WNV vector, the attenuated WNV has dual pharmacological effects of oncolysis and targeting the immune system. Its immunotherapy action and characteristics allow the attenuated WNV to extend the effective treatment period and treat tumors more effectively. As mentioned above, the immune response to the virus may cause short-term oncolytic effects of the oncolytic virus. Still, the exposure of tumor antigens+dual signals of co-stimulatory factors can activate inactive T cells in the tumor microenvironment for immune activation and immune surveillance of tumors, playing a role in the immunotherapy of tumors. At the same time, the immune memory of T-cells against tumors allows the inhibitory effect on tumors to exist for a long time, thereby being more conducive to treating recurrence, metastasis, and multiple tumors.

This invention uses the combination of immunological T-cell co-stimulatory molecules and viruses for active immunotherapy of tumors, creating a new therapeutic mechanism of action and developing a new type of RNA oncolytic virus biopharmaceutical.

The present invention also provides the virus, the vaccine, and the pharmaceutical composition, which are effective in preventing/treating liver cancer, small cell lung cancer, neurogenic tumors, neuroblastoma, neuroglioma, colorectal cancer, and leukemia.

### BRIEF DESCRIPTION OF THE DRAWINGS

The following drawings are merely examples for illustrative purposes according to various disclosed embodiments and are not intended to limit the scope of the present disclosure.

FIG. 1 illustrates array analysis of the deviation in E protein amino acids of flavivirus encephalitic viruses to JEV vaccine.

FIG. 2 illustrates schematic diagram of amino acid sites mutated for attenuation in the WNVE protein.

FIG. 3 illustrates specific targeting of attenuated WNV-Luc to neural tumors.

FIG. 4 illustrates intravenous injection of attenuated WNV shows no pathogenicity to mouse brain.

FIG. 5 illustrates schematic diagram of the genome of attenuated WNV genome, its encoded proteins, and an inserted CD86 gene.

FIG. 6 illustrates IFA (Immunofluorescence Assay) detection of virus replication in cells and expression of CD86 molecules.

FIGS. 7A, 7B, and 7C illustrate examples of significant inhibitory effect of the attenuated WNV on human neuroblastoma.

FIGS. 8A and 8B illustrate examples of significant therapeutic effect of the attenuated WNV on human small cell lung cancer.

FIG. 9 illustrates immunotherapeutic effect of attenuated WNV on mouse neuroblastoma.

FIG. 10 illustrates immunohistochemical detection of infiltrated CD4+T and CD8+T cells in the left and right tumor tissues of mice.

FIG. 11 illustrates flow cytometric analysis of T cell immune response.

### DETAILED DESCRIPTION

Recent studies have shown that WNV pathogenicity and neurotoxicity are mainly related to the E protein on the virus's surface, suggesting that attenuating its neurotoxicity, especially in the E protein, could lead to developing a safe drug or preventive vaccine for clinical application. To achieve this, we modify the envelope E protein by making nucleotide changes on several amino acids that correlate with neurotoxicity. Five amino acids were substituted: Glutamic acid at position 138 to Lysine, Tyrosine at 172 to Valine, Threonine at 173 to Alanine, Lysine at 276 to Methionine, and Alanine at 312 to Valine. Experiments demonstrated that the E gene-mutated WNV was unable to infect the central nervous system (CNS) due to its inability to pass through the blood-brain barrier. However, it retained its affinity to neural cells and could reproduce in many human and mouse cell lines, causing cell death. Moreover, the attenuated virus retained its antigenicity, which could trigger the animal's immune response and be applied as a live-attenuated WNV vaccine.

Using attenuated WNV as an oncolytic virus may have advantages over existing products.

The first is that WNV is a local epidemic agent and has not been widely vaccinated against, which has a less pre-existing immune reaction in the population in most areas, making it an attractive candidate for use as an oncolytic virus. Secondly, our study found that the neurotoxicity of WNV is related to the envelope E protein, which can be attenuated through genetic engineering. This attenuation could reduce the virus's toxicity and increase its safety as an oncolytic virus. Furthermore, unlike other oncolytic drugs such as IMLYGIC and Delytact, which are only used in treating melanoma and glioma, WNV may have cancer indications that differ from these drugs, making it a promising cancer therapy drug.

To develop oncolytic WNV, we mutated the amino acid of the WNV E protein for attenuation, and inserted a foreign gene fragment into the attenuated virus to use it as a gene therapy carrier for therapeutic drugs. Furthermore, we inserted a human CD86 gene into the attenuated WNV genome to construct and synthesize the oncolytic RNA virus, called recombinant WNV-CD86, which is marketed as Double-Spear1-H2-1 (DS1-H-21). This recombinant virus can infect tumor tissues, reproduce and kill tumor cells (oncolysis) within the tumor tissue, and simultaneously express CD86 protein molecules.

The CD86 protein is a necessary signaling molecule for activating immune T cells. It is the “master switch” for immune B cells to regulate T cell activation and tumor antigen signals. Therefore, with the help of the expression of CD86 molecules in the tumor, DS1-H2-1 has dual pharmacological effects of oncolysis and targeting the immune system. Its active immunotherapeutic effect makes DS1-H2-1 prolong the effective treatment cycle and treat tumors more effectively.

One potential issue with oncolytic viruses is that the immune response to the virus may lead to short-term effects of oncolysis. However, the dual signaling of tumor antigen plus 11nough11latory can activate inactive T-cells in the tumor microenvironment to kill the tumor. At the same time, the immune memory of the tumor makes the cancer's suppressive effect exist for a long time, which is more conducive to the treatment of tumor recurrence, metastasis, and treating multiple tumors.

So far, no oncolytic virus drugs carry CD86 active molecules in cancer immunotherapy. Therefore, the invention of the recombinant WNV-CD86 oncolytic RNA virus represents a significant breakthrough in cancer immunotherapy. It combines immunological T cell coactivating molecules and viruses to actively immunotherapy cancers, creates a novel therapeutic mechanism, and develops a new RNA oncolytic virus for the pharmaceutical industry. This discovery is expected to contribute significantly to the cure of tumor diseases and improve the lives of cancer patients.

The invention provided uses the combination of immunological T cell coactivating molecules and viruses to actively immunotherapy cancers, create a new therapeutic mechanism, and develops new RNA oncolytic virus for the pharmaceutical industry and will contribute to the cure of tumor diseases.

The present invention is illustrated below examples, but the present invention is not limited to the scope of the examples. The experimental methods in the following examples are conventional methods and conditions if not specified.

### Example 1. Mutation of the E Protein Gene Attenuates WNV Neurotoxicity

The JEV vaccine strain SA14-14-2 is originally derived from the JEV wild strain SA14. It attenuated toxicity to the central nervous system through the classic continuous passage in animal tissues and cell cultures. Comparing JEVSA14, the attenuation is due to the variation of 45 nucleotides in its genome, resulting in seventeen mutations of amino acids: one in the capsid region, eight in the E protein region, and eight in the non-structural protein region. Studies suggest that multiple mutations in the E protein region are the molecular basis for JEV virulence attenuation. Furthermore, when E protein amino acids of wild-type JEV were experimentally reversed into the attenuated YF/JEVSA14-14-2 chimera, changing the chimera to toxicity. It is proved that the envelope E protein's highly conserved amino acid sequence is crucial for attenuating neurovirulence. Among them, amino acid residue 138 (E138) of the E protein is located in the “hinge” region of the I-II interface of the E protein domain. Mutations in this region will change the three-dimensional structure of the E protein, thereby affecting the adsorption of the virus into a cell; It shows that a single reverse mutation of E138 is 12noughh to restore neurovirulence. It was required to reverse the conversion of other amino acids simultaneously, such as with E176/177 or with E279, and the neurovirulence was enabled to enhance. As an independent virulence determinant, the E176/177 cluster is located in the central domain of domain I of the E protein, which is rich in conformational epitopes sensitive to low pH; when the virus attaches to cells, it recombines with the E protein dimer as it is related to the trimeric structure with fusion activity. Residue E279, localized to E protein domain II (dimerization domain), affects neurovirulence in a manner similar to residue E138, and reverse mutation of E279 increased neurovirulence. Residue E315 localizes to the distal surface of domain III of the E protein, a region involved in attachment of virions to host cells. These experiments confirmed that the amino acid composition of E protein is related to the neurotoxicity of JEV.

WNV and JEV belong to the flavivirus genus, have the same gene structure, and encode the same functional structural and non-structural proteins. Therefore, their E protein structures are similar, but the sequences of amino acids are inconsistent. To understand the neurotoxicity of WNV E protein, we made an alignment analysis of E protein amino acids in flavivirus member. We compared the deviated amino acids of the E protein of the JEV SA14-14-2 vaccine with those at the same site of the E protein of other members of the Flavivirus. As shown in the amino acid array in FIG. 1, WNV contains the same amino acids as the JEV SA14 strain at amino acid positions 107, 138, 176-177, 244, 264, 279, and 315. However, it differs from the amino acid at the corresponding site of vaccine strain SA14-14-2. In addition, we also found that other encephalitis virus members (MVE, SLE, KUN, and JEV wild-type strains) also possessed the same amino acids at the same position but differed from the amino acid of the non-encephalitis viruses (e.g., Dengue and YFV). Through comparing, we inferred that these amino acids that are different from the E protein of the JEV vaccine strain might be related to neurotropic toxicity. Therefore, we also assumed that if these amino acid residues related to neurovirulence are replaced with the corresponding amino acids in vaccine E protein, one might reduce WNV neurotoxicity.

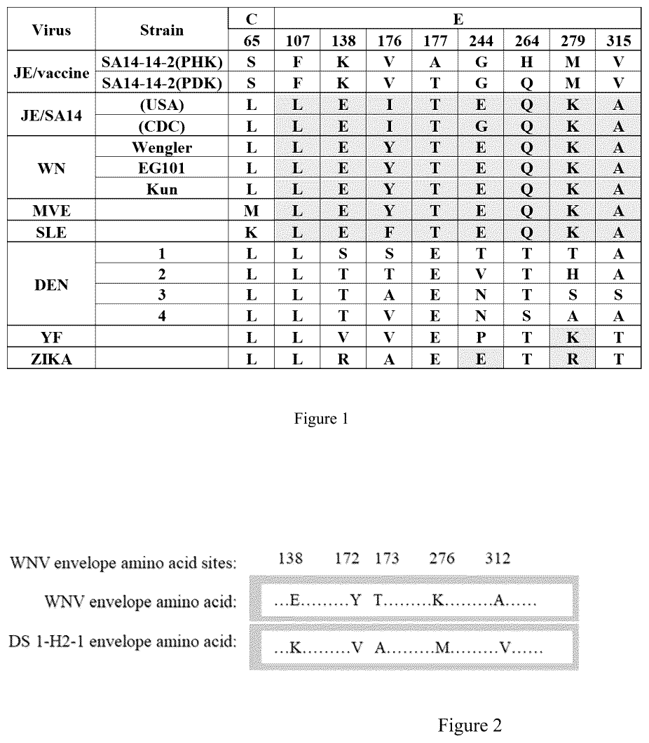

In order to verify the relationship between the amino acids of the WNV E protein and its neurotoxicity, we artificially mutated the WNV envelope E protein gene. According to the RNA sequence of the WNV B956 strain, we substituted five amino acids in the WNV E protein: Glutamine at position 138 to Lysine, Tyrosine at position 172 to Valine, Threonine at position 173 to Alanine, Lysine at position 276 to Methionine, and Alanine at position 312 to Valine (FIG. 2), and thus created CNS-attenuated WNV.

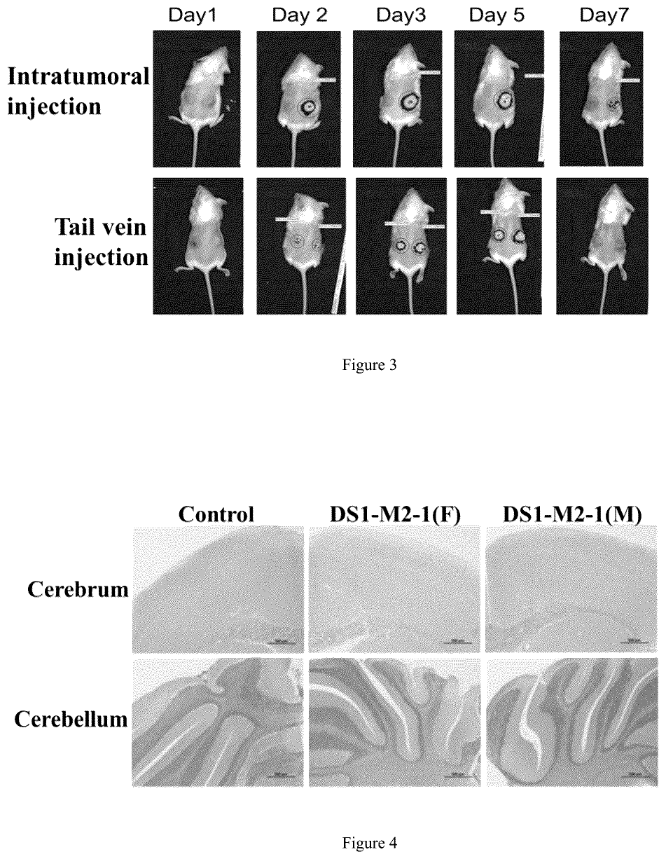

In addition, experiments proved that the synthetic attenuated WNV could replicate in various cell lines and tumor tissues but could not infect the central nervous system from peripheral administration. When injecting the attenuated WNV-carrying luciferase (1-luc2-2A-Virus) into tumor-transplanted mice through various extracranial routes, including intravenous, subcutaneous, intraperitoneal, did not cause symptoms of CNS infection in immunocompetent mice. Pharmacokinetic experiments also confirmed that the attenuated WNV, injected intravenously and intratumorally, specifically targeted to tumors, expressing luciferase proteins only in tumor tissues without entering the brain (FIG. 3), without causing CNS illness. These experiments confirmed that the five amino acids of the E protein are indeed related to neurotoxicity.

Conclusion: The five amino acids of E protein described above are indeed proven to correlate with WNV neurotoxicity. Substitution mutation of these amino acids makes it lose the ability to cross through the blood-brain barrier, and thus reduce its neurotoxicity to CNS.

### Example 2. Intravenous and Intratumoral Administration of Mice (DS1-M2-1, an Attenuated WNV Carrying the Mouse CD86 Gene) Produced an Anti-WNV Immune Response

The A/J Gpt mouse model of bilateral neuroblastoma xenografts was established. When the tumor reached the standard size (subcutaneous tumor nodule diameter 5-6 mm, 70-110 mm³), mice were randomly divided into two groups (Table 1 shows grouping and dosing information). The first administration to the right-side tumor was recorded as D1, and the administration was continued 3 times at a 3-day interval.

TABLE 1
Grouping and administration information of
intratumoral administration of DS1-M2-1

| | | | Dosing | Time and volume (mL//mouse) | | |
|---|---|---|---|---|---|---|
| Group | Mouse | injection | (PFU/mouse) | D 1 | D 4 | D 7 |
| control | 12 | Right-tumor | 0.1 mL 0.9% NaCl | 0.1 | 0.1 | 0.1 |
| DS1-M2-1 | 12 | Right-tumor | 1 × 106 | 0.1 | 0.1 | 0.1 |

During the test period, all the mice showed no abnormalities in physical examination, disease symptoms, mental state, behavioral activities, diet, or drinking water.

Mouse sera were collected on D11 and tested for anti-WNV antibodies by ELISA method. The serum of the DS1-M2-1 group was diluted 1:80, and its P/N ratio was 12.8, which was higher than 2.1 in the control group. That indicated that anti-WNV antibodies were positive in the mouse sera in the DS1-M2-1 group (Table 2).

TABLE 2
Anti-WNV antibody detection

| | Fold of | OD450 | OD450 | OD450 | |
|---|---|---|---|---|---|
| | dilution | value | negative | black | P/N* |
| Positive | 1:50 | 3.822 | 0.23 | 0.061 | + |
| sera | 1:100 | 3.692 | 0.23 | 0.061 | + |
| | 1:200 | 3.252 | 0.23 | 0.061 | + |
| | 1:400 | 2.876 | 0.23 | 0.061 | + |
| | 1:800 | 2.266 | 0.23 | 0.061 | + |
| | 1:1600 | 1.411 | 0.23 | 0.061 | + |
| Testing | 1:20 | 2.885 | 0.23 | 0.061 | + |
| sera | 1:40 | 2.528 | 0.23 | 0.061 | + |
| | 1:80 | 2.227 | 0.23 | 0.061 | + |

*P/N = (OD450 Testing sera − OD450 blank)/(OD450 negative serum − OD450 blank)

Conclusion: The production of anti-WNV antibodies indicates that the attenuated WNV induced an anti-WNV immune response, which can be applied as an attenuated-live vaccine for the prevention of infectious diseases caused by West Nile virus.

### Example 3. Intravenous Repeated Administration Toxicity Test in Mice Proves its Safety

Fourteen SPF-grade 6-week-old BALB/c mice (20-23 g) were randomly divided into 4 groups according to the principle of similar body weight an administered twice by tail vein injection. The day of administration was recorded as D1. For the specific grouping and administration, see Table 3 for drug information. The test period is 15 days.

TABLE 3
Assay for repeated administration toxicity in mice

| | Number | Virus title | Time/dosing | | |
|---|---|---|---|---|---|
| Group | (mail/femail) | (PFU/mL) | D 1, D 6 | | Results |
| Control | 5/5 | — | 0.1 | mL PBS | Normal |
| DS1-M2-1 | 5/5 | 1 × 10⁹ | 1 × 108 | PFU | Normal |

During the whole test period, all the mice in each group did not have neurological symptoms such as death, paralysis, neck stiffness, and convulsions. No apparent abnormalities were found in eating, drinking, and defecation. The mice's body weight and body temperature were normal throughout the experiment. No DS1-M2-1 virus was detected in the mice's blood collected on D4, D9, and D12. At the end of the experiment on the 15th day, the corresponding mouse tissues and organs in each group were collected and dissected. There were no apparent abnormalities in the heart, liver, spleen, lungs, kidneys, thymus, or brain tissues across all groups. Compared to the blank control group, there were no noticeable abnormalities or inflammatory changes in the mouse's brain and cerebellum tissue sections in the drug administration group (FIG. 4). That suggests that the method of repeated tail vein injection twice does not significantly impact the organs and tissues of both male and female mice tested with the attenuated WNV oncolytic drug.

Conclusion: The DS1-M2-1 with five amino acid mutations in the envelope E protein lost the ability to infect the central nervous system and has no toxicity to other tissues and organs from the peripheral administration. Therefore, WNV-CD86 is safe as an RNA virus vector drug and vaccine in clinical practice.

### Example 4. Insertion of Foreign Gene in the WNV Genome does not Affect Virus Reproduction

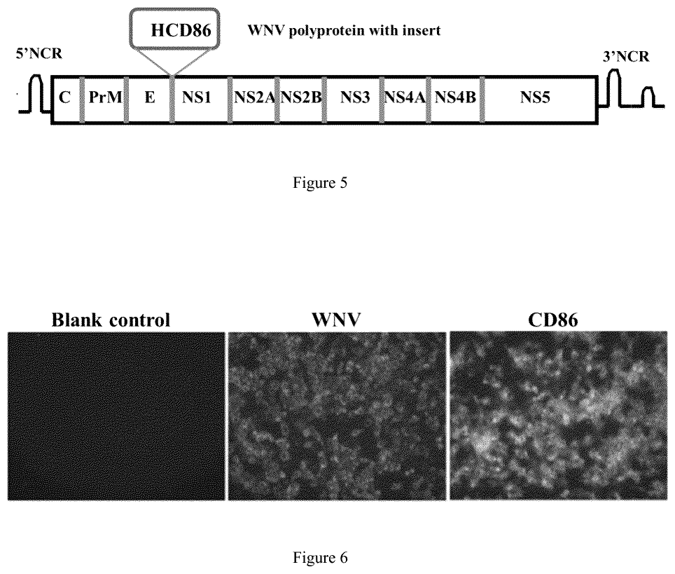

The WNV RNA genome encodes ten viral proteins (FIG. 5). When constructing and cloning the chemically synthesized WNV with E gene mutation of five amino acids into plasmid vector PBR322, a human CD86 fragment synthesized by PCR was integrated into the WNV cDNA. This human CD86 fragment is ligated (inserted) between the WNV envelope E protein and NS1 protein (FIG. 5). The PBR322 plasmid containing WNV-CD86 cDNA was transformed into Escherichia coli competent cells; from the screening transformed bacterial clones, we obtained the full-length WNV cDNA clones with mutated amino acids and with CD86 gene fragment, and sequentially acquired infectious WNV-CD86 virus by transforming the WNV cloning plasmid into cultured eukaryotic cells. The transfected Vero cells were detected by immunofluorescence with an anti-WNV-NS3 antibody and anti-HCD86 antibody, which proved that WNV plasmid DNA transcription produced the infectious virus and expressed CD86 molecule (FIG. 6). The WNV carrying the CD86 gene is named “DS1-H2-1,” for short.

Conclusion: Inserting exogenous HCD86 into the attenuated WNV genome did not affect the reproduction of the virus, and the CD86 molecule was successfully expressed. Thus, the attenuated RNA virus with the CD86 can be used as a new biological drug that acts as dual oncolytic and immunotherapy for treating tumors.

### Example 5. Oncolytic Effect of DS1-H2-1 on Human Neuroblastoma Transplanted in Mice

The attenuated WNV was tested for oncolytic virus activity. Under aseptic conditions, SH-SY5Y cells (4×106/0.1 ml/per mouse) were inoculated into the right flank region of SPF-grade BALB/c nude mice aged 8 to 10 weeks to establish a human neuroblastoma xenograft model. When the diameter of the subcutaneous tumor nodules reached 5˜6 mm (corresponding to a tumor volume of approximately 50 mm3˜100 mm3), the mice were randomly divided into groups ensuring that the difference in tumor volume between groups was <10%. Grouping and management information are as described in Table 4. The day of the first administration was recorded as D1.

TABLE 4
The experimental design and results of DS1-H2-1 in the treatment of human neuroblastoma

| | No. | Tumor Volume | Tumor | | | | ED₅₀ of |
|---|---|---|---|---|---|---|---|
| | of | (mm³) | Weight | | | | Volume |
| Group | Mice | Day 31 | (g) | % T/C | % TGI_{TV} | % TGI_{TW} | (PFU) |
| Control | 10 | 1274.20 ± 121.82 | 0.866 ± 0.142 | — | — | — | |
| Adriamycin | 10 | 468.07 ± 145.59 | 0.406 ± 0.157 * | 37.43 | 63.27 | 53.12 | |
| DS1-H2-1 | 14 | 118.35 ± 3.94 * | 0.050 ± 0.004 *** | 7.93 | 92.65 ** | 94.53 | 421 |
| (1 × 10⁶ PFU/ml) | | | | | | | |
| DS1-H2-1 | 8 | 28.39 ± 3.02 * | 0.009 ± 0.001 *** | 2.17 | 97.77 ** | 98.96 | |
| (1 × 10⁵ PFU/ml) | | | | | | | |
| DS1-H2-1 | 8 | 34.74 ± 3.95 * | 0.011 ± 0.002 *** | 2.73 | 97.27 ** | 98.73 | |
| (1 × 10⁴ PFU/ml) | | | | | | | |
| DS1-H2-1 | 8 | 35.74 ± 4.17 * | 0.016 ± 0.002 *** | 2.81 | 97.20 ** | 98.15 | |
| (1 × 10³ PFU/ml) | | | | | | | |

* P < 0.05 compared with Control group;
** P < 0.01 compared with Adriamycin group;
*** P < 0.001 compared with Control group

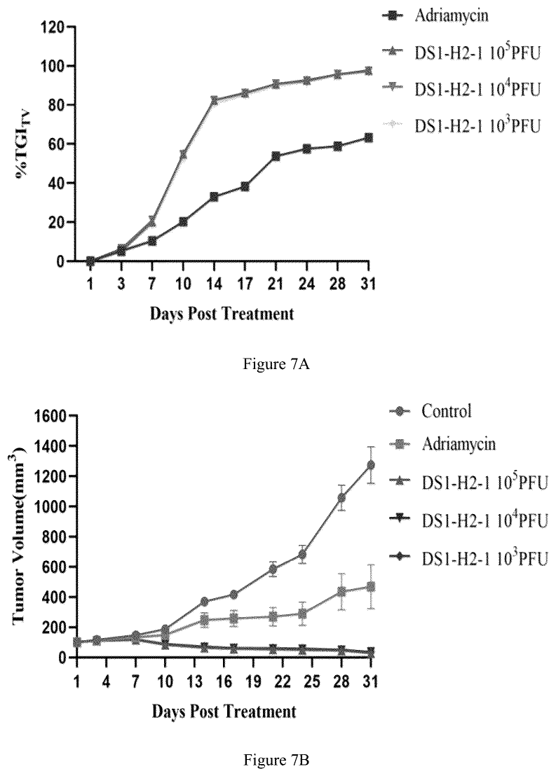

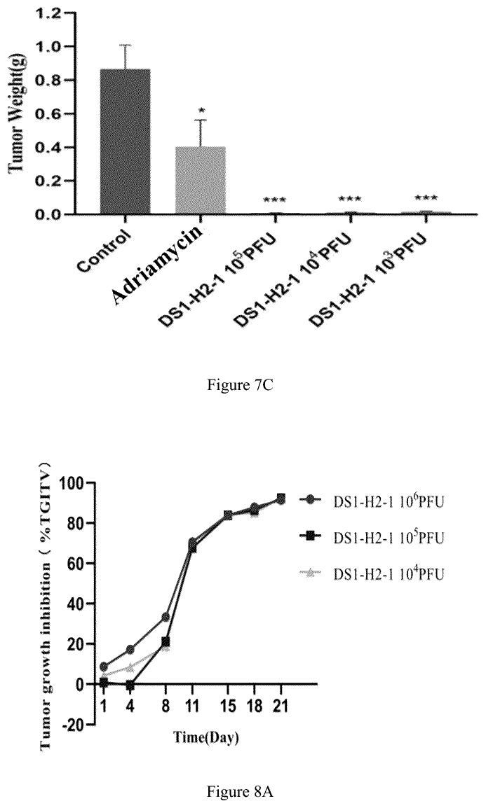

As shown in Table 4 and FIG. 7, after three times of administration (D1, D4, and D8), the tumor volume in the DS1-H2-1 group was significantly reduced, and the tumor volume shrank remarkably by four different doses. The tumor growth inhibition rate reaches 97.77% on average and is higher than the chemical drug doxorubicin (P<0.01). The half-effective dose is 421 PFU.

Conclusion: DS1-H2-1 has a significant oncolytic effect on human neuroblastoma in transplanted mice.

### Example 6. Oncolytic Therapeutic Effect of DS1-H2-1 on Human Small-Cell Lung Cancer Transplanted in Mice

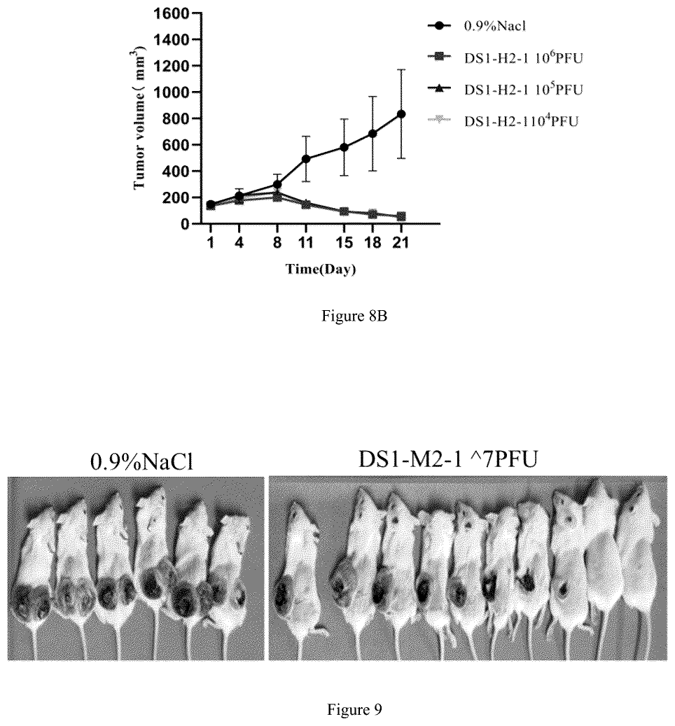

Human small cell lung cancer (NCI-H446 cells, 1×10⁸/0.1 ml/mouse) was inoculated into the right lumbar region of SPF grade 6-week-old Balb/C-nut mice (single site inoculation). When the subcutaneous tumor nodules grew to the size of 5-6 mm diameter (about 50-100 mm³), mice were randomly divided into groups per the <10% tumor volume. The grouping and drug administration information and results are shown in Table 5. The day of the first administration was recorded as D1. Results showed that three doses of DS1-H2-1 (1×10⁶ PFU, 1×10⁵ PFU, 1×10⁴ PFU) can effectively inhibit the growth and proliferation of the human small cell lung cancer transplanted in mice, especially after three administrations (D1, D4, and D8) in each treatment group, The tumor growth rate was significantly reduced, and the tumor shrank significantly (P<0.001). In addition, the tumor growth inhibition rate (% TGITV) was as high as 91.42%-92.77% on day 21 (Table 5 and FIG. 8)

TABLE 5
The experimental design and results of DS1-H2-1
in the treatment of human small-cell lung cancer

| | No. | Tumor Volume | | | | ED50 |
|---|---|---|---|---|---|---|
| | of | (mm3) | | | | of volume |
| Group | Mice | Day 1 | Day 21 | % T/C | % TGITV | (PFU) |
| Control | 6 | 148.17 ± 29.32 | 695.26 ± 308.58 | — | — | |
| Double-Spear11-H2- | 10 | 135.39 ± 10.60 | 59.64 ± 12.79 | 9.39 | 91.42 | 998.8 |
| 1 (1 × 10⁶ PFU/ml) | | | | | | |
| Double-Spear1-H2-1 | 10 | 147.07 ± 12.85 | 53.03 ± 6.62 | 7.68 | 92.37 | |
| (1 × 10⁵ PFU/m) | | | | | | |
| double spear1-H2-1 | 10 | 141.95 ± 10.03 | 50.29 ± 7.09 | 7.55 | 92.77 | |
| (1 × 10⁴ PFU/m) | | | | | | |

Conclusion: DS1-H2-1 has a significant oncolytic effect on human small-cell lung cancer transplanted in mice, and the tumor growth inhibition rate is as high as 92.77% with an ED50 of 1000 pfu.

### Example 7. The Immunotherapeutic Effect of DS1-M2-1 (Attenuated WMV Carrying Mouse CD86 Gene) on Mouse Neuroblastoma

Mouse-derived neuroblastoma cells (Neuro-2a cells, 1×10⁸/0.1 ml/mouse) were inoculated into the bilateral lumbar region of A/JGpt female mice (SPF grade 5-week-old). When the diameter of subcutaneous tumor nodules reached 5-6 mm (about 50-100 mm³), mice were randomly divided into groups per the <10% of tumor volume. The grouping and drug administration and results are shown in Table 6. The DS1-M2-1 was injected intratumorally only into the right-side tumor on day one.

TABLE 6
The experimental design and results of DS1-H2-1 in the treatment of mouse neuroblastoma.

| | No. Of mice with tumor completely eliminated | | | | | | | | | | | | Max | | ED₅₀ | |
|---|---|---|---|---|---|---|---|---|---|---|---|---|---|---|---|---|
| | D 8 | | D 11 | | D 15 | | D 18 | | D 22 | | No./Total | | % TGI_{TV} | | Log PFU | |
| Group | L | R | L | R | L | R | L | R | L | R | L | R | L | R | L | R |
| 0.9% NaCl | | | | | | | | | | | 0/15 | 0/15 | 0 | 0 | 5.8 | 4.9 |
| Doxorubicin-Hydro | | | | | | | | 1 | 3 | 2 | 3/10 | 3/10 | 84.23 | 83.25 | | |
| Taxol 20 mg/kg | | | | | | | | | | | 0/4 | 0/4 | 50.74 | 46.73 | | |
| DS1-M2-1 | 1 | 1 | 1 | 1 | | | | 3 | | 4 | 2/10 | 9/10 | 55.29 | 99.56 | | |
| 1 × 10⁷ PFU | | | | | | | | | | | | | | | | |
| DS1-M2-1 | | | 2 | 2 | | 1 | | 1 | 1 | 2 | 3/14 | 6/14 | 42.37 | 80.04 | | |
| 1 × 10⁶ PFU | | | | | | | | | | | | | | | | |
| DS1-M2-1 | | | | | 1 | | | 1 | | | 1/10 | 1/10 | 36.52 | 63.32 | | |
| 1 × 10⁴ PFU | | | | | | | | | | | | | | | | |

During the test period, the body weight of each mouse was normal. After three times of intratumoral injections of DS1-M2-1 (D1, D4, and D8), the growth of bilateral neuroblastoma in mice was effectively inhibited. The tumor volume inhibition rate on the administration side (right-side tumor) was as high as 63.32-99.56%, and the half-effective dose (ED50) of the drug was 10⁴.9 log PFU; the tumor volume inhibition rate on the non-administration side (left-side tumor) was 36.52-55.29%, and the ED50 was 10⁵.8 log PFU (Table 6). Compared with doxorubicin and paclitaxel, the right-side tumor inhibitory effect in each dose group of DS1-M2-1 was equivalent to that of doxorubicin and better than paclitaxel (FIG. 9). Since the left-side tumor is the non-administered side, it is considered that the effect of DS1-M2-1 on the left-side tumor is a result of the immunotherapy effect.

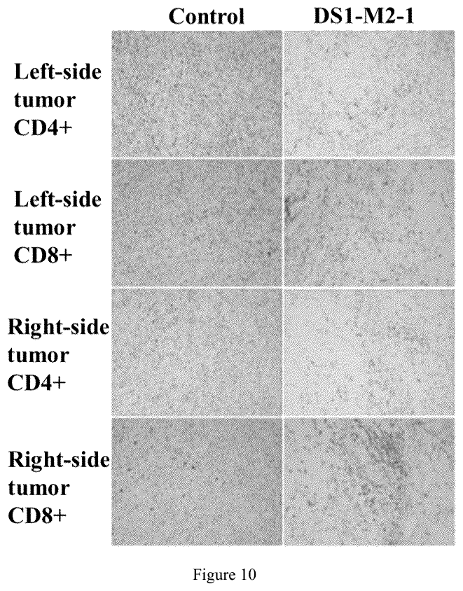

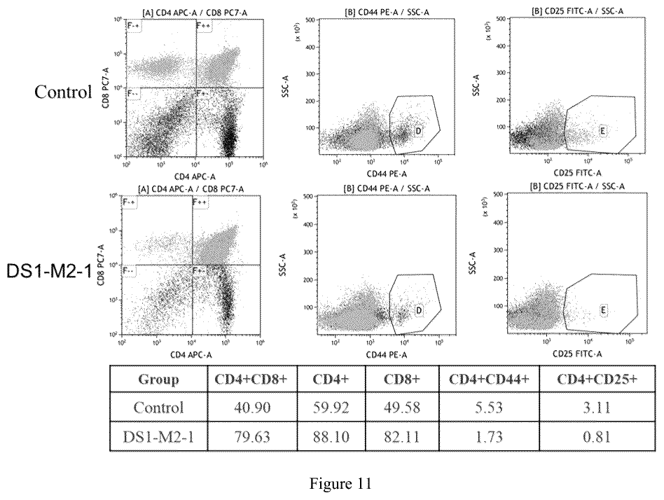

The HE staining results showed that the tumor cells in the control group grew actively, with large and deep nuclei stained and no necrosis; In contrast, the tumor tissues in the DS1-M2-1 group showed a large portion of lysed cells and necrosis with massive T-cell invasion in both, left- and right-tumors (administration and non-administration (FIG. 10). These results indicate that DS1-M2-1 induced the immune response, suggesting that DS1-M2-1 may inhibit mouse neuroblastoma through oncolysis and immunization.

Furthermore, flow cytometry analysis of T-cell immune responses revealed differences between the control and DS1-M2-1 groups. Compared to the control group, the proportion of CD4+T and CD8+ T cells in the thymus of mice in the DS1-M2-1 group increased by more than 0.5 times, while the proportion of inactivated CD4+CD44+ T and CD4+CD25+ T cells decreased by more than 3 times. All these results indicate that DS1-M2-1 induces activation of immune T cells (FIG. 11).

Conclusion: DS1-M2-1 has the therapeutic effect of oncolytic and immunotherapy on bilateral mouse neuroblastomas (proximal and distal).

The above embodiments are only used to illustrate the present invention and are not intended to limit the technical solutions described in the present invention. Therefore, the understanding of this description should be based on those skilled in the art. However, this description has described the present invention in detail with reference to the embodiments mentioned above. Note, however, those of ordinary skill in the art should understand that those skilled in the art can still modify the present invention or replace it equivalently. All technical solutions and improvements that do not depart from the spirit and scope of the present invention should be covered in within the scope of the claims of the present invention.

What is claimed is:

1. An attenuated recombinant West Nile virus, comprising:
amino acid sequence of envelope E protein of the attenuated recombinant West Nile virus has at least 98% identical to SEQ ID No: 1; and compared with the SEQ ID No: 1, the amino acid sequence of the envelope E protein has amino acid substitutions at least at positions corresponding to position 138, position 172, position 173, position 276, and position 312.

2. The attenuated recombinant West Nile virus according to claim 1, further comprising:
comparing the amino acid sequence of the envelope E protein of the virus with the SEQ ID NO: 1, the amino acid sequence of the envelope E protein has at least following mutations in a row:
a position corresponding to position 138, E is replaced by K,
a position corresponding to position 172, Y is replaced by V,
a position corresponding to position 173, T is replaced by A,
a position corresponding to position 276, K is replaced by M, and
a position corresponding to position 312, A is replaced by V.

3. The attenuated recombinant West Nile virus according to claim 1, wherein:
mutated/substituted amino acids include, but are not limited to, the presence of mutations with similar structure and properties as follows:
Glutamine (Glu/E) at position 138 is replaced with Lysine (Lys/K), arginine (Arg/R), or histidine (His/H) and other basic amino acids;
Tyrosine (Tyr/Y) at position 172 is replaced with Valine (Val/V), Methionine (Met/M), isoleucine (Ile/I), leucine (Leu/L), or Alanine (Ala/A);
Threonine (Thr/T) at position 173 is replaced with Alanine (Ala/A), Valine (Val/V), isoleucine (Ile/I), leucine (Leu/L), or Methionine (Met/M);
Lysine (Lys/K) at position 276 is replaced with Methionine (Met/M), Valine (Val/V), isoleucine (Ile/I), leucine (Leu/L), or Alanine (Ala/A);
(v) Alanine (Ala/A) at position 312 is replaced with Valine (Val/V), isoleucine (Ile/I), leucine (Leu/L), or Methionine (Met/M).

4. The attenuated recombinant West Nile virus according to claim 1, wherein:
the mutation/substitution of amino acids at positions 138, 172, 173, 276, or 312 in the envelope E protein attenuated WNV neurotoxicity and made it safe for vaccine and drug applications.

5. The attenuated recombinant West Nile virus according to claim 4, wherein:
the envelope protein mutation retained viral antigenicity and replication capacity is a good live-attenuated WNV vaccine to prevent West Nile virus diseases.

6. The attenuated recombinant West Nile virus according to claim 5, wherein:
the E protein attenuation is safely used as WNV RNA vector/oncolytic WNV drug in the treatment of cancers.

7. The attenuated recombinant West Nile virus according to claim 1, wherein:
the WNA RNA vector/oncolytic WNV embeds foreign genes, including T-cell costimulator CD86, without affecting virus replication, which targets T-cell activation for oncolytic-immunotherapy of cancers.

8. The attenuated recombinant West Nile virus according to claim 1, wherein:
a foreign non-viral gene is inserted between an envelope (E) gene and a non-structural (NS1) gene.

9. The attenuated West Nile virus according to claim 7, wherein:
the oncolytic WNV with the CD86/CD80 is used for immunotherapy of solid cancers including small-cell lung cancer, liver cancer, neuroblastoma, neuroglioma, and leukemia.


SEQUENCE LISTING (2 sequences)

SEQ ID NO: 1 - protein, 497 aa, West Nile virus
FNCLGMSNRDFLEGVSGATWVDLVLEGDSCVTLMSKDKPTIDVKMMNMEAANLADVRSYCYLASVSDLSTRAACPTMGEAHNEKRADPAFVCKQGVVDRGWGNGCGLFGKGSIDTCAKFACTTKATGWIIQKENIKYEVAIFVHGPTTVESHGKIGATQAGRFSITPSAPSYTLKLGEYGEVTVDCEPRSGIDTSAYYVMSVGAKSFLVHREWFMDLNLPWSSAGSTTWRNRETLMEFEEPHATKQSVVALGSQEGALHQALAGAIPVEFSSNTVKLTSGHLKCRVKMEKLQLKGTTYGVCSKAFKFARTPADTGHGTVVLELQYTGTDGPCKVPISSVASLNDLTPVGRLVTVNPFVSVATANSKVLIELEPPFGDSYIVVGRGEQQINHHWHKSGSSIGKAFTTTLRGAQRLAALGDTAWDFGSVGGVFTSVGKAIHQVFGGAFRSLFGGMSWITQGLLGALLLWMGINARDRSIAMTFLAVGGVLLFLSVNVHA

SEQ ID NO: 2 - protein, 497 aa, West Nile virus
FNCLGMSNRDFLEGVSGATWVDLVLEGDSCVTLMSKDKPTIDVKMMNMEAANLADVRSYCYLASVSDLSTRAACPTMGEAHNEKRADPAFVCKQGVVDRGWGNGCGLFGKGSIDTCAKFACTTKATGWIIQKENIKYKVAIFVHGPTTVESHGKIGATQAGRFSITPSAPSVALKLGEYGEVTVDCEPRSGIDTSAYYVMSVGAKSFLVHREWFMDLNLPWSSAGSTTWRNRETLMEFEEPHATKQSVVALGSQEGALHQALAGAIPVEFSSNTVMLTSGHLKCRVKMEKLQLKGTTYGVCSKAFKFARTPVDTGHGTVVLELQYTGTDGPCKVPISSVASLNDLTPVGRLVTVNPFVSVATANSKVLIELEPPFGDSYIVVGRGEQQINHHWHKSGSSIGKAFTTTLRGAQRLAALGDTAWDFGSVGGVFTSVGKAIHQVFGGAFRSLFGGMSWITQGLLGALLLWMGINARDRSIAMTFLAVGGVLLFLSVNVHA